(19) United States
(12) Patent Application Publication
(10) Pub. No.: US 2024/0156928 A1
(43) Pub. Date: May 16, 2024

(54) BI-SPECIFIC TARGETED CHIMERIC ANTIGEN RECEPTOR T CELLS

(71) Applicant: City of Hope, Duarte, CA (US)

(72) Inventors: Xiuli Wang, Duarte, CA (US); Stephen J. Forman, Duarte, CA (US); Don J. Diamond, Duarte, CA (US)

(21) Appl. No.: 18/424,395

(22) Filed: Jan. 26, 2024

Related U.S. Application Data
(63) Continuation of application No. 17/141,142, filed on Jan. 4, 2021.
Application No. 17/141,142 is a continuation of application No. 15/561,921, filed on Sep. 26, 2017, now abandoned.
(60) Provisional application No. 62/138,942, filed on Mar. 26, 2015.

(51) Int. Cl. A61K 39/00; A61K 35/17; A61K 39/12; A61K 39/17; A61K 39/245; A61P 35/00; C07K 14/705; C07K 14/71; C07K 14/725; C07K 16/28; C12N 5/0783

(52) U.S. Cl. CPC A61K 39/0011; A61K 35/17; A61K 39/12; A61K 39/17; A61K 39/245; A61P 35/00; C07K 14/70503; C07K 14/7051; C07K 14/70521; C07K 14/71; C07K 16/2803; C07K 16/2833; C12N 5/0636; C12N 5/0637; C12N 5/0638; A61K 2039/5156; A61K 2039/5158; A61K 2039/585; C07K 2317/622; C07K 2317/73; C07K 2319/03; C12N 2501/998; C12N 2710/16134

(57) ABSTRACT

T cells expressing a chimeric antigen receptor and a T cell receptor specific for CMV (bi-specific T cells) are described as a methods for using such cells in immunotherapy. In the immunotherapy methods, the recipient can be exposed to a CMV vaccine in order to expand and/or stimulate the be-specific T cells.

### CROSS REFERENCE TO RELATED APPLICATIONS

This application is a continuation of U.S. application Ser. No. 17/141,142, filed Jan. 4, 2021, and claims benefit of Ser. No. 15/561,921, filed Sep. 26, 2017, which is a U.S. National Stage Application under 35 U.S.C. § 371 and claims benefit of International Application No. PCT/US2016/024560, filed Mar. 28, 2016, which claims priority to and the benefit of U.S. Provisional Application No. 62/138,942, filed December Mar. 26, 2015. The entire contents of each of the foregoing are incorporated herein by reference, for any and all purposes.

### FEDERALLY SPONSORED RESEARCH OR DEVELOPMENT

This invention was made with government support under P50 CA107399 and P01 CA030206, awarded by the National Institutes of Health. The government has certain rights in the invention.

### SEQUENCE LISTING

This application contains a Sequence Listing that has been submitted electronically as an XML file named “40056-0020003_SL_ST26.XML.” The XML file, created on Jan. 25, 2024, is 12,539 bytes in size. The material in the XML file is hereby incorporated by reference in its entirety.

### BACKGROUND

Tumor-specific T cell based immunotherapies, including therapies employing engineered T cells, have been investigated for anti-tumor treatment. In some cases the T cells used in such therapies do not remain active in vivo for a long enough period. In some cases, the tumor-specificity of the T cells is relatively low. Therefore, there is a need in the art for tumor-specific cancer therapies with longer term anti-tumor functioning.

Adoptive T cell therapy (ACT) utilizing chimeric antigen receptor engineered (CAR) T cells may provide a safe and effective way to reduce recurrence rates of various cancers, since CAR T cells can be engineered to specifically recognize antigenically-distinct tumor populations. CAR T cells can combine the advantages of non-MHC-restricted expansion with activation and expansion of T cells. However, in some disease settings CAR therapy confers only modest clinical benefit due to attenuated persistence of CAR T cells.

### SUMMARY

Described herein are cytomegalovirus (CMV)-specific T cells that can be transduced with a chimeric antigen receptor to produce bi-specific T cells (i.e., cells specific for CMV and the antigen recognized by the CAR) useful for treating cancer patients. Subsequent to administration of the bi-specific T cell, CMV antigens (e.g., CMV peptides or cells bearing a CMV peptide) can be administered to the patient. This CMV peptide vaccination can promote proliferation of the bi-specific T cells and enhance their anti-tumor activity. The CAR expressed by the bi-specific T cells can be any CAR, for example, a CAR targeted to CD19, CD123 or HER2. In some cases the T cells do not recognize an antigen from a second virus. For example, they do not recognize an Epstein-Barr virus antigen or a Influenza virus antigen or an Adenovirus antigen.

Described herein is a method for preparing T cells specific for cytomegalovirus (CMV) (e.g., a population of T cells comprising cells specific for a variety of different CMV antigens) and expressing a chimeric antigen receptor (CAR) (e.g., a CAR that binds a cancer antigen), the method comprising: (a) providing PBMC from a cytomegalovirus (CMV)-seropositive human donor; (b) exposing the PBMC to at least one CMV antigen; (c) treating the exposed cells to produce a population of cells enriched for stimulated cells specific for CMV (e.g., treating them to create a population of cells that is enriched for cells stimulated cells specific for CMV relative to the untreated population of cells); (d) transducing at least a portion of the enriched population of cells with a vector (e.g., a lentiviral vector) expressing a CAR, thereby preparing T cells specific for CMV and expressing a CAR.

In various cases: the step of treating the exposed cells (e.g., using a selection step) to produce a population of cells enriched for stimulated cells specific for CMV comprises treating the stimulated cells to produce a population of cells enriched for cells expressing an activation marker (e.g., IFN-7 of IL-13); the PBMC are cultured for less than 5 days (less than 4, 3, 2, 1 days) prior to exposure to the CMV antigen; the cells are exposed to the CMV antigen for fewer than 3 days (fewer than 48 hrs, 36 hrs, 24 hrs) the CMV antigen is pp65 protein or an antigenic portion thereof, the CMV antigen comprises two or more different antigenic CMV pp65 peptides; the step of transducing the enriched population of cells does not comprise CD3 stimulation; the step of transducing the enriched population of cells does not comprise CD28 stimulation; the step of transducing the enriched population of cells does not comprise CD3 stimulation or CD28 stimulation; the enriched population of cells is at least 40% (e.g., 50%, 60%, 70%) IFN-γ positive, at least 20% (e.g., 25%, 30%, 35%) CD8 positive, and at least 20% (e.g., 25%, 30%, 35%) CD4 positive; the enriched population of cells are cultured for fewer than 10 (fewer than 9, 8, 7, 5, 3, 2) days prior to the step of transducing the enriched population of cells with a vector encoding a CAR. In some cases PBMC are from a CMV positive donor are exposed to a CMV antigen such as CMV pp65 or a mixture of CMV protein peptides (for example 10-20 amino acid peptides that are fragments of pp65) in the presence of IL-2 to create a population of stimulated cells. In some cases the population of stimulated cells is treated to prepare a population of cells that express IFN-7.

In some case the method further comprises expanding the CMV specific T cells expressing a CAR cells by exposing them an antigen that binds to the CAR.

In some case the step of expanding the CMV-specific T cells expressing a CAR comprises exposing the cells to T cells expressing the antigen that bind the CAR (e.g., the expansion takes place is the presence of at least one exogenously added interleukin (e.g., one or both of IL-1 and IL-15) and a T cell expressing the antigen recognized by the CAR.

In various cases: the CAR is selective for an antigen selected from: CD19, CS1, CD123, 5T4, 8H9, αvβ6 integrin, alphafetoprotein (AFP), B7-H6, CA-125 carbonic anhydrase 9 (CA9), CD19, CD20, CD22, CD30, CD33, CD38, CD44, CD44v6, CD44v7/8, CD52, CD123, CD171, carcionoembryonic antigen (CEA), EGFrvIII, epithelial glycoprotein-2 (EGP-2), epithelial glycoprotein-40 (EGP-40), ErbB1/EGFR, ErbB2/HER2/neu/EGFR2, ErbB3, ErbB4, epithelial tumor antigen (ETA), FBP, fetal acetylcholine receptor (AchR), folate receptor-α, G250/CAIX, ganglioside 2 (GD2), ganglioside 3 (GD3), HLA-A1, HLA-A2, high molecular weight melanoma-associated antigen (HMW-MAA), IL-13 receptor a2, KDR, k-light chain, Lewis Y (LeY), L1 cell adhesion molecule, melanoma-associated antigen (MAGE-A1), mesothelin, Murine CMV infected cella, mucin-1 (MUC1), mucin-16 (MUC16), natural killer group 2 member D (NKG2D) ligands, nerve cell adhesion molecule (NCAM), NY-ESO-1, Oncofetal antigen (h5T4), prostate stem cell antigen (PSCA), prostate-specific membrane antigen (PSMA), receptor-tyrosine kinase-like orphan receptor 1 (ROR1), TAA targeted by mAb IgE, tumor-associated glycoprotein-72 (TAG-72), tyrosinase, and vascular endothelial growth factor (VEGF) receptors.

In some cases the CAR is selective for an antigen selected from: CD19, CD123, CS1, BCMA, CD44v6, CD33, CD22, IL-13α2, PSA, HER2, EGFRv3, CEA, and C7R.

In some cases: the CAR comprises: a scFv selective for the selected non-CMV antigen; a hinge/linker region; a transmembrane domain; a co-signaling domain; and CD3 (signaling domain; the chimeric antigen receptor further comprises a spacer sequence located between the co-signaling domain and the CD3ζ signaling domain; the co-signaling domain is selected from a CD28 co-signaling domain and a 4-IBB co-signaling domain; the transmembrane domain is selected from a CD28 transmembrane domain and a CD4 transmembrane domain; the vector expressing the CAR expresses a truncated human EGFR from the same transcript encoding the CAR, wherein the truncated human EGFR lacks a EGF ligand binding domain and lacks a cytoplasmic signaling domain; the spacer sequence comprises or consists of 3-10 consecutive Gly; the hinge/linker region comprises at least 10 amino acids of an IgG constant region or hinge region; the IgG is IgG4; the hinge/linger region comprises an IgG4 CD3 domain; the hinge/linger region comprises an IgG4 Fc domain or a variant thereof, the hinge/linker region comprises or consists of 4-12 amino acids; and hinge/linker region is selected from the group consisting of: the sequence ESKYGPPCPPCPGGGSSGGGSG (SEQ ID NO:18) and the sequence GGGSSGGGSG (SEQ ID NO:7).

Also described herein is population of human T cells specific for CMV and transduced by a vector comprising an expression cassette encoding a chimeric antigen receptor, wherein at least 20% of the cells in the population are CD4+, at least 20% of the cells in the population are CD8+ and at least 60% of the cells in the population are IFNγ+.

In various cases: the T cells are specific for CMV pp65; and the CAR binds an antigen selected from: CD19, CD123, CS1, BCMA CD44v6, CD33, CD22, IL-13a2, PSA, HER2, EGFRv3, CEA, and C7R.

Also described is a method of treating a patient suffering from cancer comprising administering a composition comprising bi-specific cells. In various cases: the population of human T cells are autologous to the patient; the population of human T cells are allogenic to the patient; the population of human T cells are autologous to the patient; the method further comprises administering to the patient a CMV antigen; the step of administering a CMV antigen comprising administering T cells loaded with a CMV antigen or a mixture of CMV antigens (for example pp65 peptide or mixture of 10-20 amino acid peptides that are fragments of pp65); the T cells loaded with a CMV antigen are autologous to the patient; and the step of exposing the patient to a CMV antigen comprises exposing the patient to antigen presenting cells bearing a CMV antigen.

T cells expressing a CAR targeting CD19 can be useful in treatment of cancers such as B cell lymphomas, as well as other cancer that expresses. Thus, this disclosure includes methods for treating cancer using T cells expressing a CAR described herein.

This disclosure also includes methods for making the bi-specific T cells and methods of using the bi-specific T cells to treat patients.

An “amino acid modification” refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

### Components of Chimeric Antigen Receptors

A wide variety of CAR have been described in the scientific literature. In general CAR include an extracellular antigen-binding domain (often a scFv derived from variable heavy and light chains of an antibody), a spacer/linker domain, a transmembrane domain and an intracellular signaling domain. The intracellular signaling domain usually includes the endodomain of a T cell co-stimulatory molecule (e.g., CD28, 4-1BB or OX-40) and the intracellular domain of CD3ζ.

### Hinge/Linker Region

In certain embodiments, the hinge/linger is derived from an IgG1, IgG2, IgG3, or IgG4 that includes one or more amino acid residues substituted with an amino acid residue different from that present in an unmodified hinge. The one or more substituted amino acid residues are selected from, but not limited to one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof.

In some embodiments, the modified hinge is derived from an IgG1, IgG2, IgG3, or IgG4 that includes, but is not limited to, one or more of the following amino acid residue substitutions: C220S, C226S, S228P, C229S, P230S, E233P, V234A, L234V, L234F, L234A, L235A, L235E, G236A, G237A, P238S, S239D, F243L, P2471, S267E, H268Q, S280H, K290S, K290E, K290N, R292P, N297A, N297Q, S298A, S298G, S298D, S298V, T299A, Y300L, V3051, V309L, E318A, K326A, K326W, K326E, L328F, A330L, A330S, A331S, P331S, 1332E, E333A, E333S, E333S, K334A, A339D, A339Q, P396L, or a combination thereof.

In some embodiments, the modified hinge is derived from a human IgG4 hinge/CH2/CH3 region having the following amino acid sequence (e.g., is at least 90%, at least 95%, at least 98% identical to or identical to):
ESKYGPPCPS CPAPEFLGGP SVFLFPPKPK DTLMISRTPE VTCVVVDVSQ EDPEVQFNWY 219
VDGVEVHNAK TKPREEQFNS TYRVVSVLTV LHQDWLNGKE YKCKVSNKGL PSSIEKTISK 279
AKGQPREPQV YTLPPSQEEM TKNQVSLTCL VKGFYPSDIA VEWESNGQPE NNYKTTPPVL 339
DSDGSFFLYS RLTVDKSRWQ EGNVFSCSVM HEALHNHYTQ KSLSLSLGK (SEQ ID NO:1)

In certain embodiments, the modified hinge is derived from IgG4 that includes one or more amino acid residues substituted with an amino acid residue different from that present in an unmodified hinge. The one or more substituted amino acid residues are selected from, but not limited to one or more amino acid residues at positions 220, 226, 228, 229, 230, 233, 234, 235, 234, 237, 238, 239, 243, 247, 267, 268, 280, 290, 292, 297, 298, 299, 300, 305, 309, 218, 326, 330, 331, 332, 333, 334, 336, 339, or a combination thereof.

In some embodiments, the modified hinge is derived from an IgG4 that includes, but is not limited to, one or more of the following amino acid residue substitutions: 220S, 226S, 228P, 229S, 230S, 233P, 234A, 234V, 234F, 234A, 235A, 235E, 236A, 237A, 238S, 239D, 243L, 2471, 267E, 268Q, 280H, 290S, 290E, 290N, 292P, 297A, 297Q, 298A, 298G, 298D, 298V, 299A, 300L, 3051, 309L, 318A, 326A, 326W, 326E, 328F, 330L, 330S, 331S, 331S, 332E, 333A, 333S, 333S, 334A, 339D, 339Q, 396L, or a combination thereof, wherein the amino acid in the unmodified hinge is substituted with the above identified amino acids at the indicated position. In one instance the sequence includes the following amino acid changes S228P, L235E and N297Q.

For amino acid positions in immunoglobulin discussed herein, numbering is according to the EU index or EU numbering scheme (Kabat et al. 1991 Sequences of Proteins of Immunological Interest, 5th Ed., United States Public Health Service, National Institutes of Health, Bethesda, hereby entirely incorporated by reference). The EU index or EU index as in Kabat or EU numbering scheme refers to the numbering of the EU antibody (Edelman et al. 1969 Proc Natl Acad Sci USA 63:78-85).

The hinge/linker region can also comprise a IgG4 hinge region having the sequence ESKYGPPCPSCP (SEQ ID NO:2) or ESKYGPPCPPCP (SEQ ID NO:3).

The hinge/linger region can also comprise the sequence ESKYGPPCPPCP (SEQ ID NO:3) followed by the linker sequence GGGSSGGGSG (SEQ ID NO:7) followed by IgG4 CH3 sequence GQPREPQVYTLPPSQEEMTKNQVSLTCLVKGFYPSDIAVEWESNGQPENNYKTTP PVLDSDGSFFLYSRLTVDKSRWQEGNVFSCSVMHEALHNHYTQKSLSLSLGK (SEQ ID NO:8). Thus, the entire linker/spacer region can comprise the sequence: ESKYGPPCPPCPGGGSSGGGSGGQPREPQVYTLPPSQEEMTKNQVSLTCLVKGFYP SDIAVEWESNGQPENNYKTTPPVLDSDGSFFLYSRLTVDKSRWQEGNVFSCSVMH EALHNHYTQKSLSLSLGK (SEQ ID NO:9). In some cases the linker/space has 1, 2, 3, 4, or 5 single amino acid changes (e.g., conservative changes) compared to SEQ ID NO:9. In some cases, the IgG4 Fc hinge/linker region that is mutated at two sites within the CH2 region (L235E; N297Q) in a manner that reduces binding by Fc receptors (FcRs).

### Transmembrane Region

In some cases the transmembrane region is a CD4 transmembrane region, e.g., having region having the following amino acid sequence (e.g., is at least 90%, at least 95%, at least 98% identical to or identical to): MALIVLGGVAGLLLFIGLGIFF (SEQ ID NO:10). In some cases the transmembrane region is a CD28 transmembrane region, e.g., having region having the following amino acid sequence (e.g., is at least 90%, at least 95%, at least 98% identical to or identical to): MFWVLVVVGGVLACYSLLVTVAFIIFWV (SEQ ID NO:11)

### Co-Signaling Domain

The co-signaling domain can be any domain that is suitable for use with a CD3ζ signaling domain. In some cases the co-signaling domain is a CD28 co-signaling domain that includes a sequence that is at least 90%, at least 95%, at least 98% identical to or identical to: RSKRSRGGHSDYMNMTPRRPGPTRKHYQPYAPPRDFAAYRS (SEQ ID NO:12). In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:13).

### CD3ζ Signaling Domain

The CD3ζ Signaling domain can be any domain that is suitable for use with a CD3ζ signaling domain. In some cases the co-signaling domain is a CD28 co-signaling domain that includes a sequence that is at least 90%, at least 95%, at least 98% identical to or identical to: RSKRSRGGHSDYMNMTPRRPGPTRKHYQPYAPPRDFAAYRS (SEQ ID NO:14).

### DESCRIPTION OF DRAWINGS

FIGS. 1A-D depict the development of clinically feasible platform for derivation of bi-specific T cells and the schematic structure of a lentiviral vector expressing a CD19 CAR. (A) CMV-specific T cells from CMV immune HLA A2 donors were selected using IFNγ capture after overnight stimulation with cGMP grade CMVpp65 protein. After selection, the cells were stained with antibodies specific to IFNγ, CD4, and CD8. The frequency of each population is presented after exclusion of dead cells with DAPI. (B) The selected cells were transduced with the second generation CD19CAR with a double mutation in the spacer, 24 hours after the IFNγ capture. 7-10 days later, the transduced cells were stimulated with irradiated CD19 expressing NIH3T3 cells at 10:1 ratio (3T3: T cells) and the stimulation was repeated 7 days post the first stimulation. CAR expression was defined by cetuximab-biotin and streptavidin (SA) APC-Cy7 staining. Percentages of CAR⁺ cells are indicated in each histogram (filled gray), and based on subtraction of that stained with SA-APC-Cy7 alone (black line). (C) Growth of total cell number was determined by Guava Viacount at different time points. (D) Schematic diagram of 10039 nt lentiviral vector encoding a CD19 CAR. Within the 3183 nucleotide long CD19R:CD28:z(CO)-T2A-EGFRt construct, the CD19-specific scFv, IgG4 Fc spacer, theCD28 transmembrane and cytoplasmic signaling domains, three-glycine linker, and CD3z cytoplasmic signaling domains of the CD19R:CD28:z(CO) CAR containing the 2 point mutations, L235E and N297Q, in the CH2 portion of the IgG4 spacer (CD19R(EQ)), as well as the T2A ribosome skip and truncated EGFR sequences are indicated. The human GM-CSF receptor alpha signal sequences that drive surface translocation of the CD19R:CD28:z(CO) CAR and EGFRt are also indicated.

FIGS. 2A-2C depict the results of studies demonstrating that bi-specific T cells exhibit specific effector function after engagement with CD19⁺ and CMVpp65⁺ tumors. (A) 7 days after the second CD19 Ag stimulation, T cells were stained with HLA A2 restricted pp65 tetramer, cetuximab-biotin, anti-CD8 and antibodies specific to central memory T cell surface markers. Percent positive cells are indicated after dead cell exclusion with DAPI, gating based on pp65 tetramer and cetuximab double-positivity, and isotype-matched stained samples. (B) Four-hour ⁵¹Cr release assays were performed using the bi-specific T cells and indicated ⁵¹Cr-labeled target cells at different effector: target (E:T) ratios. OKT3-expressing LCLs were used as positive controls, KG1A and U251T as negative controls. CD19⁺ LCL and engineered pp65U251T cells were used as target for CD19 and CMV-specific T cells, respectively. Data from a representative donor is presented. (C) Bi-specific T cells (10⁵) were activated overnight with 10⁵ LCL-OKT3, LCL, or KG1a in 96-well tissue culture plates and 10⁵ U251T and engineered pp65 expressing U251T cells (pp65U251T) in 24-well tissue culture plates. Supernatants were collected after overnight co-incubation of bi-specific T cells and stimulators. Cytokine levels with indicated stimulators (means±SEM of triplicate wells) were determined using cytometric bead array.

FIG. 2D depicts the results of studies examining cytokine levels in the serum of bi-specific T cell treated tumor bearing mice. NSG mice were injected i.v. on day 0 with 2.5×106 GFPffluc+ LCL cells. Three days after tumor inoculation, recipient mice were administered i.v. with 2×106 bi-specific cells that underwent 2 rounds of CD19 stimulation. Vaccine was given by i.v. injection of peptide (pp65 or MP1) pulsed autologous T cells on day 14. Thirteen days post vaccine, serum of recipient mice was collected and levels of human cytokines were determined by cytometric bead array. Cytokine levels in the serum of untreated mice was used as baseline. Mean and SEMs from triplicates are presented.

FIGS. 3A-3B depict the results of studies demonstrating that bi-specific T cells exhibit bi-effector function after stimulation through TCR and CAR. (A) pp65 tetramer analysis of expanded bi-specific T cells was performed before and after each CD19 Ag stimulation by flow cytometry. Percentages of pp65 tetramer and CD8 double-positive cells are indicated based on negative tetramer and isotype gating. (B) Bi-specific T cells (10⁵) were activated overnight with 10′ of LCL-OKT3, LCL, KG1a in 96-well tissue culture plates and 10⁵ U251T and engineered pp65 expressing U251T cells (pp65U251T) in 24-well tissue culture plates. Co-cultures were fixed and permeabilized using the BD Cytofix/Cytoperm kit according to manufacturer's instructions. After fixation and permeabilization, the T cells were stained with anti-IFNγ. Before fixation, anti-cetuximab-biotin and anti-CD3 staining was used to analyze surface expression of CAR and T cells. Percentages of positive cells on gated CD3 T cells are presented based on that stained with isotype antibodies.

FIGS. 4A-4B depict the results of studies demonstrating that bi-specific T cells proliferate after re-stimulation through TCR and CAR. Bi-specific T cells isolated by IFNγ capture and stimulated with two cycles of CD19 Ag were labeled with CFSE and co-cultured with indicated stimulators for 8 days. (A) CFSE retention on gated live T cells is shown. (B) Quantification of CFSE retention of CAR⁺ T cells. Subtractions of percentages and mean fluorescence intensity (MFI) of CFSE expression of negative control KG1a to LCL and U251T to pp65U251T are depicted.

FIGS. 5A-5C depict the results of studies demonstrating that anti-tumor activity of adoptively transferred bi-specific T cells is enhanced by CMVpp65 vaccination. (A) NSG mice were injected i.v. on day 0 with 2.5×10⁶ GFPffluc⁺ LCL cells. Three days after tumor inoculation, recipient mice were injected i.v. with 2×10⁶ bi-specific cells that underwent 2 rounds of CD19 stimulation. Vaccine was given by i.v. injection of peptide pulsed autologous T cells. Fourteen to seventeen days post T cell infusion, 5×10⁶ pp65pepmix (B) or pp65 peptide (C) (or MP1) loaded autologous T cells were irradiated and injected (iv) into T-cell-engrafted mice as vaccine. pp65 vaccine was also supplemented to the mice that were treated with 10×10\hat{ }6 CMV-specific T cells from the same donor and untreated mice were used as another type of control. Tumor growth was evaluated by Xenogen® imaging. N=5 for each group in the experiments. The Mann Whitney test was used for statistical analysis.

FIG. 5D shows the results of studies demonstrating enhanced anti-tumor activity of adoptively transferred bi-specific T cells by CMV vaccine for replased tumor After stimulation with cGMP grade CMVpp65 protein, the CMV specific T cells were transduced with lenti-viral vector expressing CD19RCD28EGFRt and re-stimulated with irradiated CD19+ expressing tumor for 2 cycles. 5×10\hat{ }6 bi-specific T cells were injected (i.v) into CD19+ LCL bearing NSG mice. When tumor relapsed on day 18 5×10\hat{ }6 pp65peptide (or MP1) loaded autologus T cells were irradiated and injected (i.v) into T cell engrafted mice as vaccine. Tumor signals were monitored by xenogen imaging.

FIGS. 6A-6D depict the results of studies demonstrating that adoptively transferred bi-specific T cells can be expanded via CMVpp65 vaccine and ablated by cetuximab. 2×10⁶ CMV-specific or bi-specific T cells from the same donor were adoptively transferred into CD19 tumor-bearing NSG mice. 2 weeks post T cell infusion, mice received either pp65 vaccine or MP1 vaccine. (A) Percentages of human T cells pooled from blood, bone marrow and spleen from multiple mice (N=4) and (B) GFP⁺ tumor cells in the mouse spleen were determined by flow cytometry. The Mann Whitney test was used for statistical analysis. (C) CMVpp65 tetramer and CAR double positive cells in the spleen of mice were analyzed by flow cytometry after labeling with antibodies specific to human CD45, pp65 tetramer and EGFR, 28 days post bi-specific T cell infusion. The percentages of CMVpp65 tetramer⁺ CAR⁺ T cells in the human T cell population of a representative mouse are presented. (D) 1×10⁶ bi-specific T cells were adoptively transferred into CD19 tumor-bearing NSG mice. 2 weeks post T cell engraftment, mice received cetuximab (Erbitux™) 1 mg/day i.p. injection for 4 days. One day after the last injection, CD45⁺GFP⁻ human T cells and CD45⁺CAR⁺ T cells in the bone marrow were analyzed by flow cytometry after staining with antibodies specific to human CD45 and cetuximab-biotin. Representative FACS data from cetuximab-treated and untreated mice are depicted on the left and percentages of CAR⁺ T cells in the mouse bone marrow from multiple mice are presented on the right.

FIG. 7 is a Schematic of the CD19CAR-T2A-EGFRt lentiviral vector Diagram of the cDNA open reading frame of the 3183 nucleotide long CD19R:CD28:z(CO)-T2A-EGFRt construct, where the CD19-specific scFv, IgG4 Fc spacer, CD28 transmembrane and cytoplasmic signaling, three-glycine linker, and CD3z cytoplasmic signaling domains of the CD19R:CD28:z(CO) CAR containing the 2 point mutations, L235E and N297Q, in the CH2 portion of the IgG4 spacer (CD19R(EQ)), as well as thT2A ribosome skip and truncated EGFR sequences are indicated. The human GM-CSF receptor alpha signal sequences that drive surface translocation of the CD19R:CD28:z(CO) CAR and EGFRt are also indicated.

### DETAILED DESCRIPTION

Described below are T cells specific for CMV and CD19. These bi-specific T cells were generated using a rapid and efficient method for generating and selecting CMV-specific T cells. The method, which employs IFNγ capture of CMV-specific T cells, consistently and efficiently enriched CMV-specific T cells while preserving the broad spectrum of CMV repertoires. Moreover, the cells remained amenable to gene modification after a brief CMVpp65 stimulation, avoiding the need for CD3/CD28 bead activation prior to transduction. This is significant because CD3/CD28 activation can cause activation-induced cell death (AICD) of CMV-specific T cells (30). Engineering the bulk IFNγ-captured T cells with a CD19CAR lentivirus followed by stimulation with CD19 antigen resulted in 50 to 70% of the CAR⁺ T cells responding to pp65 stimulation, representing the subset of functional bi-specific T cells. The bi-specific T cells exhibited specific cytolytic activity and secreted IFNγ, as well as proliferating vigorously after engagement of endogenous CMVpp65 T cell receptors or engineered CD19 CARs. Upon transfer into tumor bearing mice, the bi-specific T cells mediated cytokine released syndrome (CRS), which has been found to correlate with anti-tumor efficacy in the clinic (2, 31). Importantly, the methods described herein are capable of generating therapeutic doses of functional bi-specific T cells within 3-4 weeks, ensuring timely production for clinical application.

Efficient in vivo activation of virus-specific T cells through the TCR demands that viral antigens are processed and presented in a human leukocyte antigen (HLA)-dependent manner. In the mouse model studies described below, we generated APC by loading autologous T cells with either pp65 peptide or a full-length pp65pepmix. The effects of vaccination were indistinguishable whether using pp65 peptide or pp65 pepmix. Both approaches elicited bi-specific T cell responses and induced enhanced antitumor activity compared with irrelevant MP1 challenge. The response of bi-specific T cells to vaccine might be even more efficient in immunocompetent patients, where more professional APC are present than in these immunocompromised mouse studies.

The studies described below demonstrate that the antitumor activity of bi-specific CMV/CD19 T cells can be enhanced as a consequence of proliferation following CMV peptide vaccination. This suggests that the cell dose of bi-specific T cells could be significantly decreased as compared to conventional CD19CAR T cells, due to their potential to proliferate in vivo in response to vaccine, avoiding prolonged culture times and the risk of terminal differentiation. Potential on/off-target toxicity can potentially be controlled by ablation of infused CAR T cells using cetuximab. These results illustrate the clinical applications of CMV vaccine to augment the antitumor activity of adoptively transferred CD19CAR T cells in several scenarios: 1) to salvage patients not achieving complete remission or relapsing after CAR T cell therapy, 2) vaccine boost when CD19 CAR T cells are failing to persist regardless of tumor responses at that time, 3) planned vaccination on days 28 and 58 post-CD19 CAR T cells, which has been shown an effective immune-stimulation in our CMV peptide vaccine. There is also potential benefit of using the bi-specific T cells pre-emptively post-allogeneic HCT, both to eliminate minimal residual disease (MRD) and control CMV, potentially preventing reactivation of virus or undergoing expansion in response to latent CMV re-activation.

Moreover, this CMV vaccine strategy has the potential to profoundly impact the general field of adoptive T cell therapy, since by transducing a variety of tumor-directed CARs into our CMV-specific T cells, we have the potential to tailor this strategy to a wide range of malignancies and tumor targets.

Enrichment of CMV-Specific T Cells from PBMC of Healthy Donors after Stimulation with cGMP Grade CMVpp65 Protein

CMV-specific T cells were prepared from PBMC of healthy donors by stimulating the PBMC with cGMP grade CMVpp65 protein. Briefly, PBMCs were isolated by density gradient centrifugation over Ficoll-Paque (Pharmacia Biotech, Piscataway, NJ) from peripheral blood of consented healthy, HLA-A2 CMV-immune donors under a City of Hope Internal Review Board-approved protocol. PBMC were frozen for later use. After overnight rest in RPMI medium containing 5% Human AB serum (Gemini Bio Products) without cytokine, the PBMC were stimulated with current good manufacturing practice (cGMP) grade CMVpp65 protein (Miltenyi Biotec, Germany) at 10 ul/10×10⁶ cells for 16 hours in RPMI 1640 (Irvine Scientific, Santa Ana, CA) supplemented with 2 mM L-glutamine (Irvine Scientific), 25 mM N-2-hydroxyethylpiperazine-N-2-ethanesulfonic acid (HEPES, Irvine Scientific), 100 U/mL penicillin, 0.1 mg/mL streptomycin (Irvine Scientific) in the presence of 5U/ml IL-2 and 10% human AB serum. CMV-specific T cells were selected using the IFNγ capture (Miltenyi Biotec, Germany) technique according to the manufacturer's instructions.

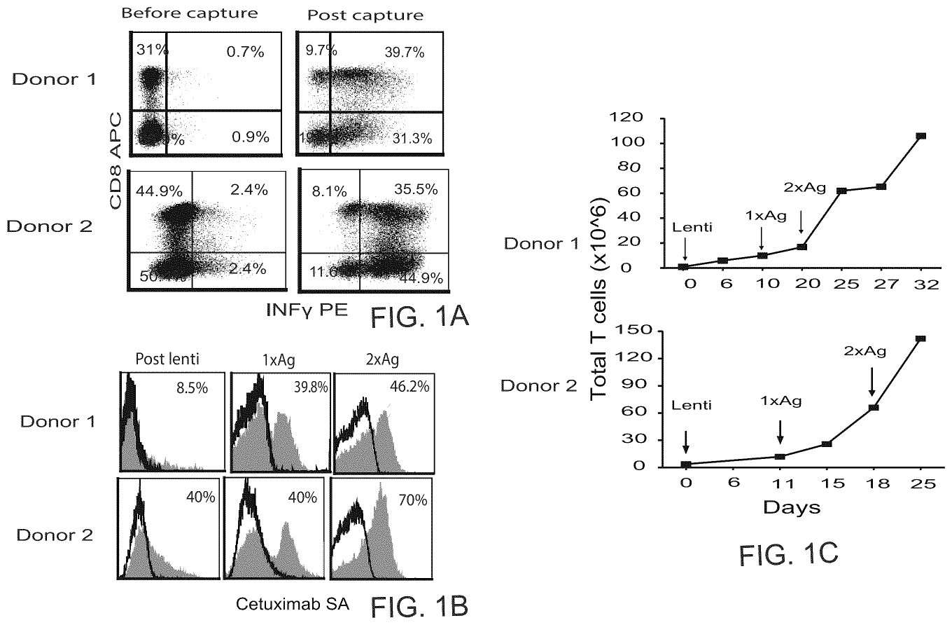

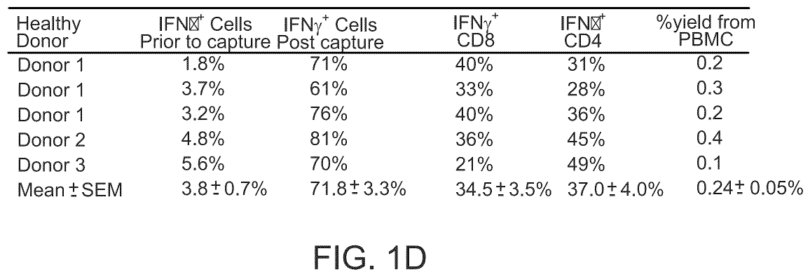

To demonstrate the consistency of this clinically feasible process, the selection was repeated five times using PBMC from three different donors. IFNγ-positive T cells were consistently enriched from a baseline mean of 3.8% (range 1.8-5.6) to a post-capture mean of 71.8% (range 61-81) and contained polyclonal CD8⁺ (34%) and CD4⁺ T cells (37%) after selection (FIG. 1A and FIG. 1C). Moreover, the selected CMV-specific T cells included both CD4 and CD8 subsets and represented the entire spectrum of CMV-specificity, showing responsiveness to CMVpp65 pepmix stimulation with broad recognition.

### Genetic Modification of Enriched CMV-Specific T Cells to Express CD19 CAR and In Vitro Expansion of the Bi-Specific T Cells

In the clinically adaptable procedure, IFNγ-captured CMV-specific T cells were transduced 2 days after the selection, without OKT3 activation, using the second generation CD19RCD28EGFRt lentiviral construct containing the IgG4 Fc hinge region mutations (L235E; N297Q) that we have determined to improve potency due to distortion of the FcR binding domain (21, 22). Starting seven days post lenti-transduction, the cells were stimulated on a weekly basis with 8000 cGy-irradiated, CD19-expressing NIH3T3 cells at a 1:10 ratio (T cells: CD19NIH 3T3). The percentage of CAR⁺ cells detected by cetuximab increased from 8% post transduction to 46% after 2 rounds of stimulation with a 120-150-fold total cell increase (FIG. 1B and FIG. 1D). Further details regarding the lentiviral construct, the CD19-expressing NIH3T3 cells and other materials and techniques used in the studies described herein are presented below.

Bi-Specific T Cells Exhibited Specific Effector Function after Stimulation Through Pre-Defined Viral TCR and CD19CAR

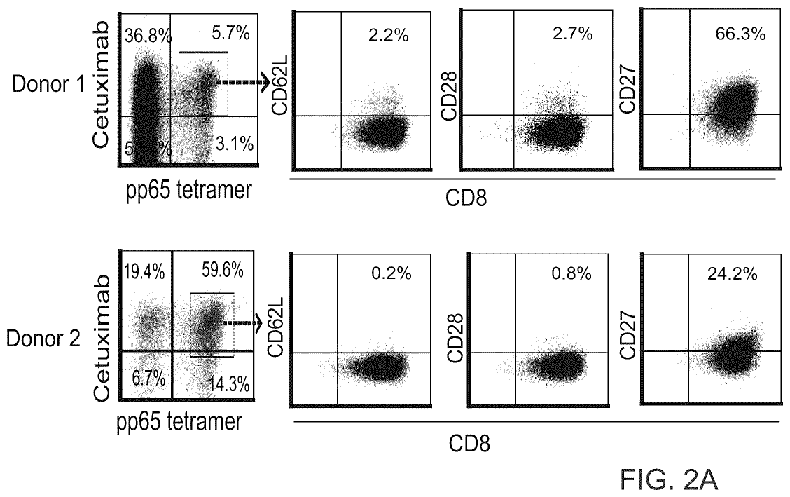

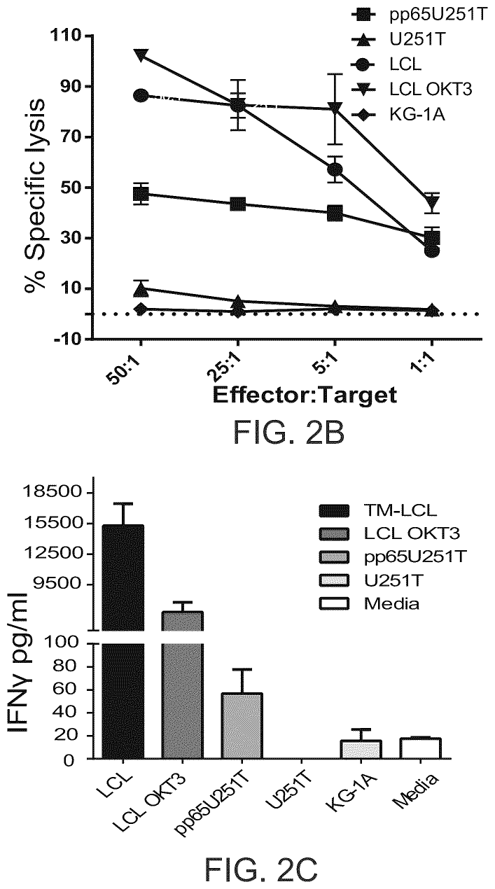

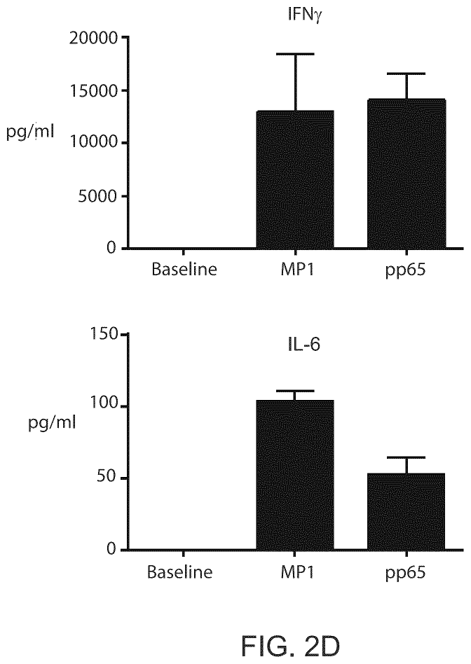

Recapitulating our previous studies (23), the ex vivo expanded CMV-specific T cells possessed an effector phenotype and no longer expressed the central memory markers of the originally selected cells, such as CD62L, CD28, and IL-7Ra (FIG. 2A and FIG. 2D). However, levels of CD27 remained high, suggesting a greater proliferative potential that has been associated with greater clinical efficacy (24). To investigate bi-specific T cell effector function via signaling by both the endogenous CMV-specific TCR and the introduced CD19CAR, we evaluated response to engineered pp65-expressing U251T cells from HLA-A2 donors, and also allogeneic CD19⁺LCLs, based on cytotoxicity, cytokine production and proliferation profiles. As expected, the expanded bi-specific T cells specifically lysed CD19⁺LCLs with the same maximum killing levels as the OKT3-expressing LCL used as positive controls. Likewise, specific killing was also observed when pp65U251T cells were used as targets as compared to parental U251T cells (FIG. 2B). Accordingly, after overnight stimulation, elevated IFNγ secretion was observed after either CD19 or pp65 antigen stimulation as compared to antigen-negative stimulators such as KG1a and U251T parental cells (FIG. 2C).

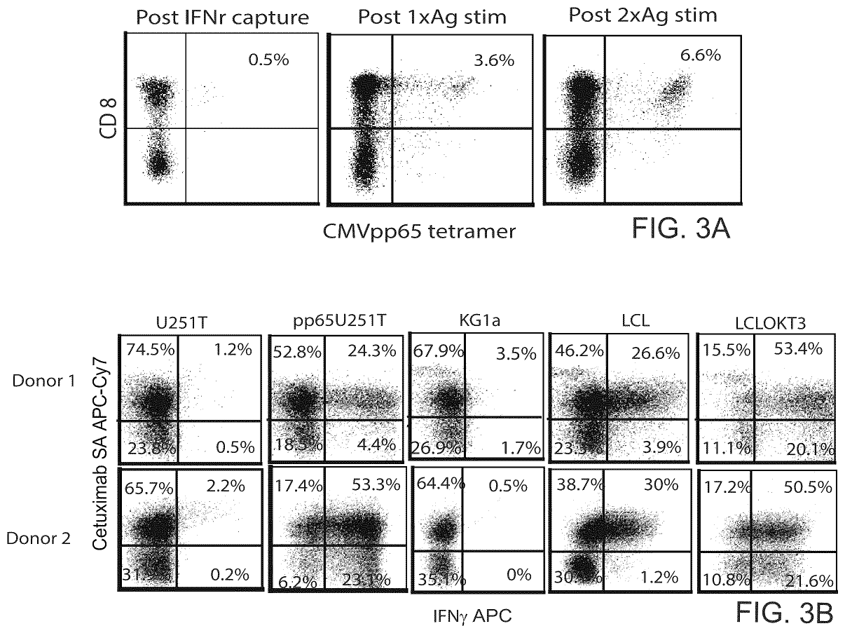

Although CMV-specific T cells were enriched prior to lentiviral transduction, the T cell population is mixed, including CMV-specific T cells, CD19CAR⁺ T cells, bi-specific T cells, and possibly a small percentage of T cells that are neither CMV-specific nor CD19CAR⁺. T cell expansion following lentiviral transduction is predominantly CD19-driven through CAR stimulation, so we next investigated how CAR stimulation affects the composition of CMV-specific T cells. Using pp65 tetramer as an indicator of the CMV-specific population, we found that the percentage of CMVpp65 tetramer-positive cells increased from 0.5% to 6.6% by the end of the second CD19 stimulation, indicating bi-specific T cells proliferated strongly with CD19 stimulation (FIG. 3A).

To further investigate that these effector functions were attributable to bi-specific T cells rather than distinct CD19CAR⁺ and CMV-specific T cell subsets in the population, we performed intracellular cytokine (ICC) assays. In response to pp65 antigen stimulation, 24-53% of the T cells in the population were CAR⁺ and able to secret IFNγ (FIG. 3B). ˜30% of T cells exhibited IFNγ secretion upon stimulation with CD19⁺ LCL cells.

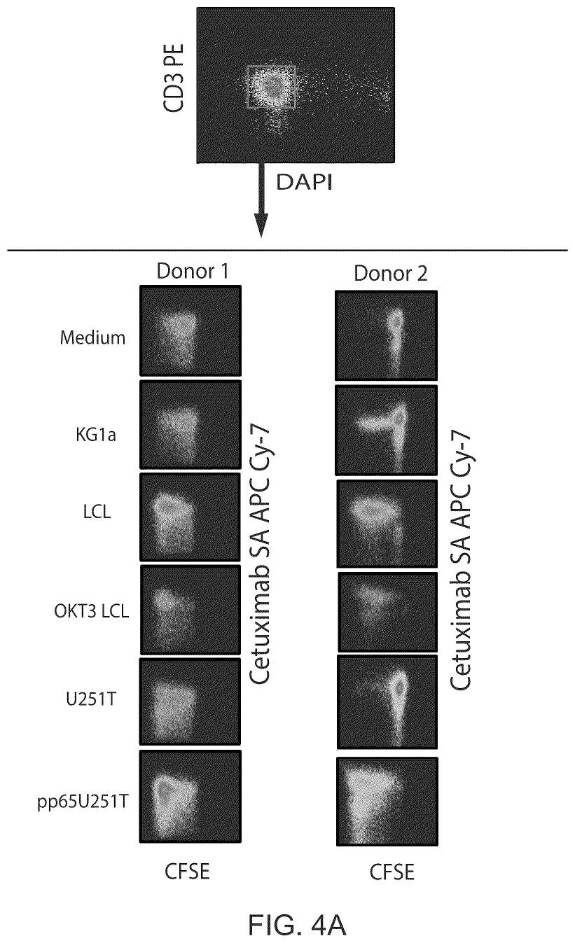

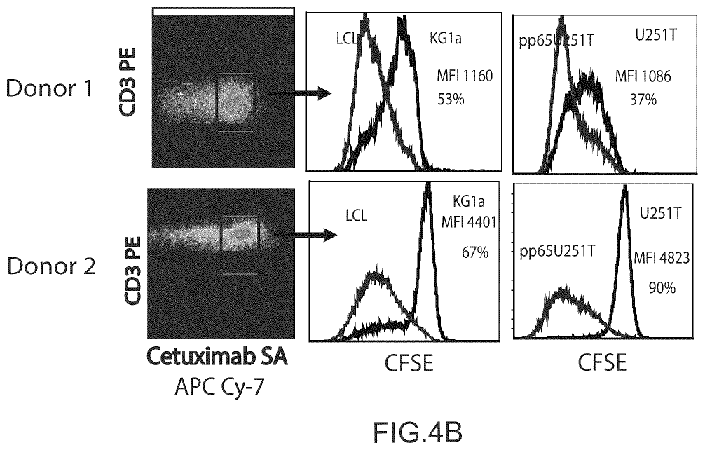

To assess the ability of bi-specific T cells to proliferate in response to CD19 or pp65 antigen stimulation, T cells were labeled with CFSE and co-cultured with different stimulators, and then evaluated for CFSE dilution 8 days later. Unlike the cultures stimulated with CD19-negative KG1a and U251T cells, cell division was more robust after stimulation through either the CD19 CAR⁺ (LCL cells) or the CMV-specific TCR (pp65U251T cells) (FIG. 4). Building on these findings, we next performed in vivo experiments to examine the effects of CMV peptide vaccine on the expansion and anti-lymphoma efficacy of adoptively transferred bi-specific T cells.

Anti-Lymphoma Activity of Adoptively Transferred Bi-Specific T Cells was Augmented In Vivo by Vaccination with CMVpp65 Peptide Antigen

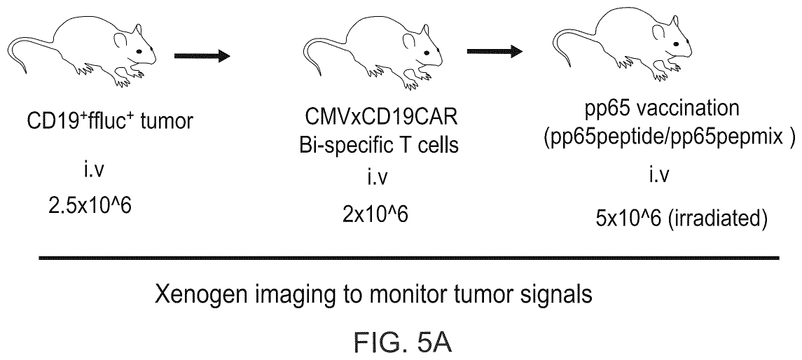

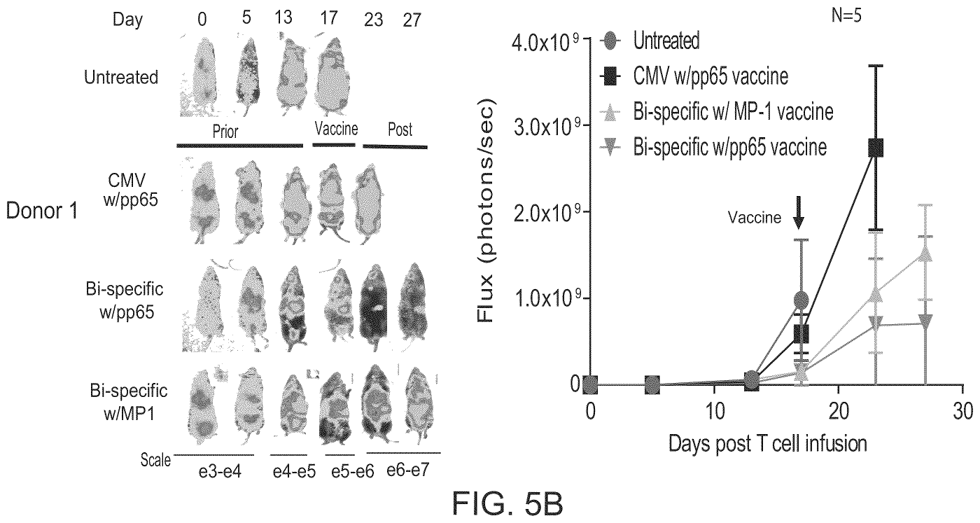

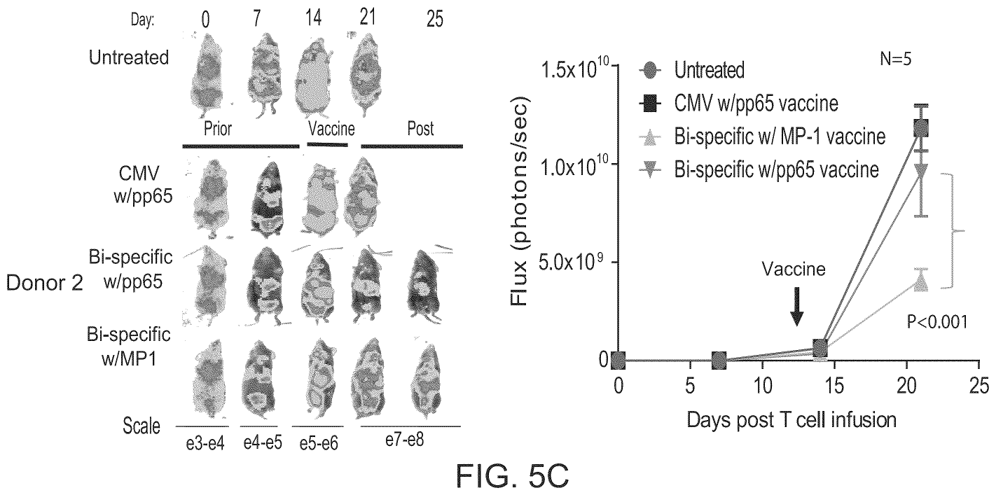

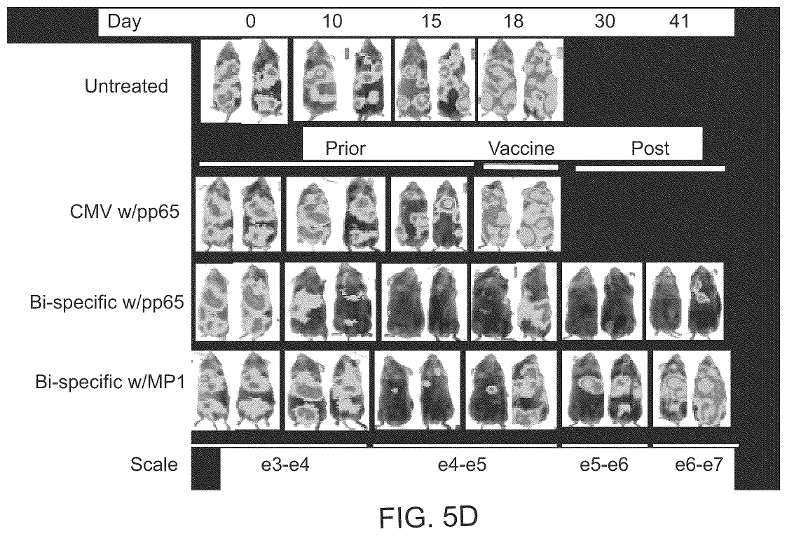

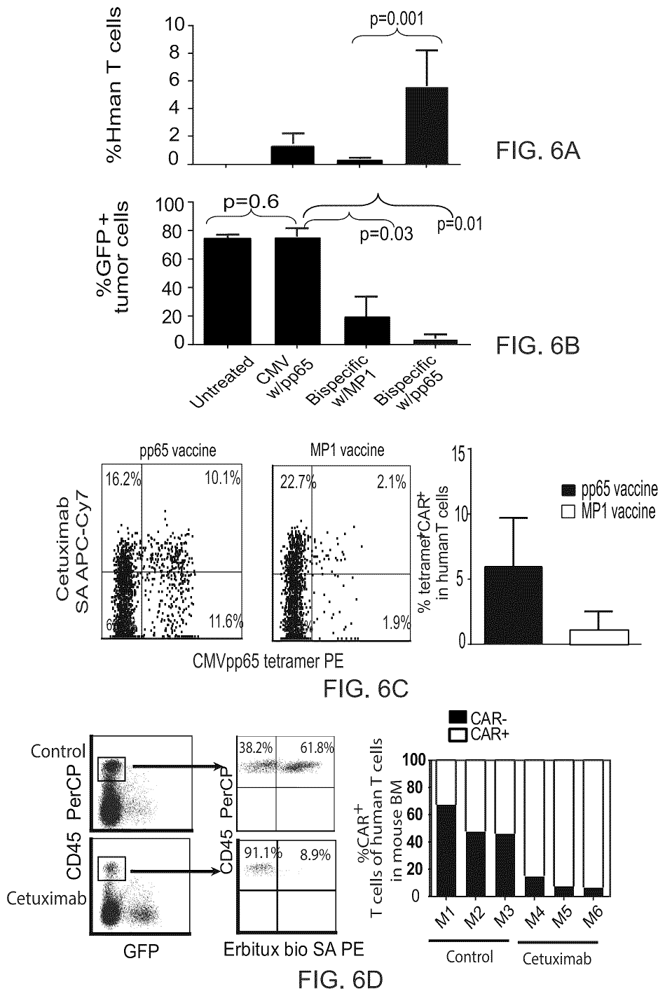

Our preliminary studies have demonstrated that engineered CD19CAR T cells can target and lyse CD19 positive lymphoma in vivo. However, the antitumor efficacy is suboptimal and tumor reduction represents a transient event followed by eventual tumor progression (data not shown) unless high doses of CAR T cells were infused (21). In this study, we wanted to tease out the differences between the targeted and control vaccines. Therefore we chose a suboptimal T cell dose (1×10⁶ CAR T cells), which is 10 times lower than the curative dose we used previously (10×10⁶) (21). We attempted to augment antitumor efficacy using a CMV peptide vaccine boost (FIG. 5A). As expected, as few as 2×10⁶ bi-specific T cells were able to induce a specific tumor reduction as compared to untreated and CMV-mono-specific T cell treated mice. We observed augmented anti-tumor activity after vaccination with pp65-peptide-pulsed T-APC of two different formulations [pp65pepmix (FIG. 5B) and HLA A2-restricted CMVpp65 peptide (FIG. 5C)], but not in mice that were vaccinated using T-APC loaded with the irrelevant peptide MP1 (HLA A2 restricted). Interestingly, mice that received bi-specific T cell treatment had to be euthanized around the same time as control mice even though the tumor signals were dramatically lower (FIG. 5). Our further studies indicated that there were highly elevated levels of human specific IFNγ and IL-6 in the mouse serum (55) and it is probable that the mice died of cytokine release syndrome (25) rather than tumor. More interestingly, augmented anti-tumor efficacy induced by pp65 vaccine was supported in a relapsed tumor model. To further demonstrate that the enhanced anti-lymphoma activity is attributable to expansion of bi-specific T cells in response to CMVpp65 stimulation, human T cells and CAR⁺ Tetramer⁺ T cells harvested from mice were analyzed 10-14 days after vaccination. As expected, human T cells in the mice treated with bi-specific T cells were significantly higher in the pp65-challenged mice (5.6±2.6%) than in MP1 controls (0.3±0.1%) (FIG. 6A). The levels of human T cells and bi-specific T cells were well correlated with the tumor reduction based on GFP expression by FACS analysis (FIG. 6B). Further, CAR⁺ and CMVpp65 tetramer⁺ bi-specific T cells harvested from mice were more abundant in the pp65 peptide-challenged mice than in MP1 controls (FIG. 6C). However, pp65 Tetramer/CAR+ double positive cells were only detected in the spleen, possibly indicating a unique homing characteristic of the population of bi-specific T cells. In addition to the pre-defined viral TCR that can be used to boost antitumor activity in vivo through peptide vaccine, functional bi-specific T cells are also expected to proliferate upon exposure to CD19 antigen in vivo. This was supported by the finding that there were lower levels of engraftment of CMV-specific T cells as compared to bi-specific T cells in tumor-bearing mice, even though the same pp65 peptide vaccine was used to stimulate both types of T cells (FIG. 6A). These data suggested that bi-specific T cells were able to proliferate and expand in vivo in response to stimulation of the TCR as well as the CD19 CAR.

### Adoptively Transferred Bi-Specific T Cells are Efficiently Ablated by Cetuximab-Mediated Antibody Dependent Cell Mediated Cytotoxicity (ADCC) In Vivo

The impressive clinical efficacy of CAR T cell therapy and the frequently associated on/off-target toxicities such as cytokine release syndrome (CRS), have highlighted the need for T cell ablation strategies (1, 3, 4, 26). Taking advantage of the properties of the EGFRt receptor translated from the same transcript as the CD19CAR, we tested the anti-EGFR monoclonal antibody cetuximab for its ability to ablate CAR⁺ T cells. Fourteen days after engrafting mice with bi-specific T cells, cetuximab was administered intraperitoneally at 1 mg/day for 4 consecutive days. CAR⁺ cells in the bone marrow were significantly decreased as compared to untreated mice. 50-60% of human T cells are CAR⁺ in the bone marrow of untreated controls, however, less than 10% of the human T cells in cetuximab treated mice are CAR⁺ (FIG. 6D), suggesting successful ablation (68% CAR T cell elimination) based on antibody binding to the EGFRt.

The studies described above that examined the extent to which bi-specific T cells eradicate tumors in NSG mice revealed that a few tumor cells remained after mice were treated with bi-specific T cells and pp65 vaccine; in contrast, many more tumor cells were detected in the mice receiving only un-engineered CMV-specific T cells—the same percentage as was seen in untreated controls, and in the mice that received bi-specific T cells without pp65 vaccine (FIG. 6B). Consistently, expansion of bi-specific T cells was much lower in mice that received an irrelevant MP1vaccine compared to those that received pp65 vaccine (2% vs 10%), further demonstrating the specificity of the response to vaccination in bi-specific T cell-treated animals. Meanwhile, we noticed that the percentage of pp65⁺/CAR⁺ double-positive human cells harvested from mice were much decreased compared to the input human T cell population. We speculate that the tetramer-negative population has disproportionately expanded in vivo compared to tetramer-positive cells, since this subset includes cells expressing mouse xeno-reactive native T cell receptors. It is also possible that another contribution to the decline in the proportion of pp65⁺/CAR⁺ cells from the input population could be a result of these double-positive cells undergoing activation-induced cell death (AICD) after killing tumor cells, due to their effector T cell characteristics (FIG. 2A). AICD could be thought of as a deleterious effect of the vaccine on pp65⁺/CAR⁺, but could actually be a measure of effectiveness as demonstrated by decreased tumor burden (FIG. 6B). Ongoing studies on the functional responses to CMV vaccine of the different T cell subsets of the infused product will further reveal the mechanisms of the enhanced antitumor activity.

Pre-clinical studies with engineered CAR T cells in different xenotransplant tumor models have demonstrated variable potency with some showing tumor eradication in the short window tested and some reporting eventual tumor relapse (17, 22, 32, 33). Several variables of these artificial systems, such as the aggressiveness of the tumor cell line, tumor burden at the time of CAR T cell infusion, dose of CAR T cells may account for perceived differences in CAR potency, making it difficult to compare between xenograft models. Optimal growth signals are required for efficient and sustained expansion of transfused effector T cells in vivo. These signals encompass T-helper cell interactions, native TCR/CD3 complex signaling, and the activation of costimulatory signals. Although the CAR is designed to mimic the TCR and transmit activation signaling, the lack of in vivo persistence of some CAR T cells has been attributed to incomplete stimulation after engagement of the CAR (8, 10). This study suggests that the interaction of CAR T cells with tumor cells is inadequate to completely eradicate the transplanted tumor. This could be a result of insufficient growth signal transmission through the CAR for T cell expansion and activation, or insufficient cytolytic activation of T cells to kill tumor targets. T cell activation through viral TCRs has several advantages over self antigen TCR in promoting robust T cell expansion. Signaling through a viral TCR is generally far more robust than through a self-antigen specific TCR, since the viral-specific TCR affinity to antigen has not been dampened by the effects of tolerance and negative selection (34). A recent study is emblematic of the contrast in T cell activation caused by stimulation through a self antigen such as p53 and the immune response to antigens expressed from a viral vector (35). Since the viral TCR is expressed from the same cell as the CAR, the robust T cell activation caused by an antiviral TCR could lead to enhanced antitumor activity as a consequence of the expansion of CMV-specific CAR T cells.

Efficiently controlling proliferation to avoid cytokine storm and off-target toxicity is an important hurdle for the success of T cell immunotherapy. The EGFRt incorporated in the CD19CAR lentiviral vector will serve not only as a marker for detection and selection of CAR T cells, but may also act as suicide gene to ablate the CAR⁺ T cells in cases of treatment-related toxicity. In this study, bi-specific T cell engrafted mice were treated with cetuximab daily for 4 days. Consequently, more than 68% of the persistent CAR⁺ T cells were ablated in NSG mice as a result of ADCC, CDC and direct killing by cetuximab (36), despite the lack of professional ADCC effectors such as NK and B cells in the NSG mouse model. More efficient ablation is expected in humans, in the presence of a full panel of effector cells.

Antibodies and Flow Cytometry: Fluorochrome-conjugated isotype controls, anti-CD3, anti-CD4, anti-CD8, anti-CD28, anti-CD45, anti-CD27, anti-CD62L, anti-CD127, anti-IFN□, and streptavidin were obtained from BD Biosciences. Biotinylated cetuximab was generated from cetuximab purchased from the City of Hope pharmacy. The IFN-□ Secretion Assay—Cell Enrichment and Detection Kit and CMVpp65 protein were purchased from Miltenyi Biotec (Miltenyi Biotec, Germany). Phycoerythrin (PE)-conjugated CMV pp65 (NLVPMVATV; SEQ ID NO: 16)-HLA-A2*0201 iTAg MHC tetramer, PE-conjugated multi-allele negative tetramer was obtained from Beckman Coulter (Fullerton, CA). Carboxyfluorescein diacetate succinimidyl ester (CFSE) was purchased from Invitrogen (Carlsbad, CA). All monoclonal antibodies, tetramers and CFSE were used according to the manufacturer's instructions. Flow cytometry data acquisition was performed on a MACSQuant (Miltenyi Biotec, Germany) or FACScalibur (BD Biosciences), and the percentage of cells in a region of analysis was calculated using FCS Express V3 (De Novo Software).

Cell lines: EBV-transformed lymphoblastoid cell lines (LCLs) were made from peripheral blood mononuclear cells (PBMC) as previously described (16). To generate LCL-OKT3, allogeneic LCLs were resuspended in nucleofection solution using the Amaxa Nucleofector kit T, OKT3-2A-Hygromycin_pEK plasmid was added to 5 μg/107 cells, the cells were electroporated using the Amaxa Nucleofector I, and the resulting cells were grown in RPMI 1640 with 10% FCS containing 0.4 mg/ml hygromycin. To generate firefly luciferase+ GFP+ LCLs (fflucGFPLCLs), LCLs were transduced with lentiviral vector encoding eGFP-ffluc. Initial transduction efficiency was 48.5%, so the GFP+ cells were sorted by FACS for >98% purity. To generate CD19 NIH3T3 cells, parental NIH3T3 cells (ATCC) were transduced with a retrovirus encoding CD80, CD54 and CD58 (17). The established cell line was further engineered to express CD19GFP by lentiviral transduction. GFP+ cells were purified by FACS sorting and expanded for the use of stimulation of bi-specific T cells. To generate pp65 stimulator cells, U251T cells derived from human glioblastoma cells from an HLA A2 donor (ATCC) were transduced with a lentiviral vector encoding full length pp65 fused to green fluorescent protein (GFP). pp65U251T cells were purified by GFP expression using flow cytometry. Banks of all cell lines were authenticated for the desired antigen/marker expression by flow cytometry prior to cryopreservation, and thawed cells were cultured for less than 6 months prior to use in assays.

Peptides: The pp65 peptide NLVPMVATV (SEQ ID NO:16) (HLA-A 0201 CMVpp65) at >90% purity was synthesized using automated solid phase peptide synthesis in the TVR (Beckman Research Institute of City of Hope). MP1 GIGFVFTL (SEQ ID NO:17) peptide (HLA-A 0201 influenza) was synthesized at the City of Hope DNA/RNA Peptide Synthesis Facility, (Duarte, CA). pepMix HCMVA (pp65) (pp65pepmix) was purchased from JPT peptide Technologies (GmbH, Berlin Germany). All peptides were used according to the manufacturer's instructions.

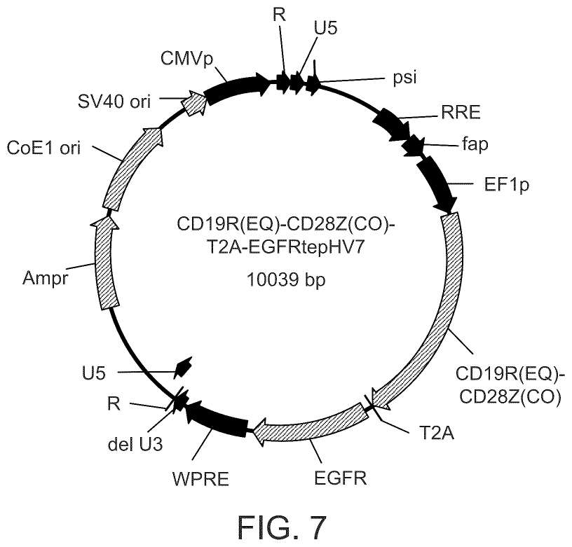

Lentivirus vector construction: The lentivirus CAR construct was modified from the previously described CD19-specific scFvFc:ζ chimeric immunoreceptor(18), to create a third-generation vector. The CD19CAR containing a CD28Q co-stimulatory domain carries mutations at two sites (L235E; N297Q) within the CH2 region on the IgG4-Fc spacers to ensure enhanced potency and persistence after adoptive transfer (FIG. 7). The lentiviral vector also expressed a truncated human epidermal growth factor receptor (huEGFRt), which includes a cetuximab (Erbitux™) binding domain but excludes the EGF-ligand binding and cytoplasmic signaling domains. A T2A ribosome skip sequence links the codon-optimized CD19R:CD28:ζ sequence to the huEGFRt sequence, resulting in coordinate expression of both CD19R:CD28:ζ and EGFRt from a single transcript (CD19CARCD28EGFRt) (19). The CD19RCD28EGFRt DNA sequence (optimized by GeneArt) was then cloned into a self-inactivating (SIN) lentiviral vector pHIV7 (gift from Jiing-Kuan Yee, Beckman Research Institute of City of Hope) in which the CMV promoter was replaced by the EF-1α promoter.

Enrichment of CMV-specific T cells after CMVpp65 protein stimulation: PBMCs were isolated by density gradient centrifugation over Ficoll-Paque (Pharmacia Biotech, Piscataway, NJ) from peripheral blood of consented healthy, HLA-A2 CMV-immune donors under a City of Hope Internal Review Board-approved protocol. PBMC were frozen for later use. After overnight rest in RPMI medium containing 5% Human AB serum (Gemini Bio Products) without cytokine, the PBMC were stimulated with current good manufacturing practice (cGMP) grade CMVpp65 protein (Miltenyi Biotec, Germany) at 10 μl/10×10⁶ cells for 16 hours in RPMI 1640 (Irvine Scientific, Santa Ana, CA) supplemented with 2 mM L-glutamine (Irvine Scientific), 25 mM N-2-hydroxyethylpiperazine-N′-2-ethanesulfonic acid (HEPES, Irvine Scientific), 100 U/mL penicillin, 0.1 mg/mL streptomycin (Irvine Scientific) in the presence of 5U/ml IL-2 and 10% human AB serum. CMV-specific T cells were selected using the IFNγ capture (Miltenyi Biotec, Germany) technique according to the manufacturer's instructions.

Derivation and expansion of bi-specific T cells: The selected CMV-specific T cells were transduced on day 2 post IFNγ capture with lentiviral vector expressing CD19CARCD28EGFRt at MOI 3. Seven to ten days after lenti-transduction, the bi-specific T cells were expanded by stimulation through CAR-mediated activation signals using 8000 cGy-irradiated CD19-expressing NIH 3T3 cells at a 10:1 ratio (T cells:CD19 NIH3T3) once a week as described (17) in the presence of IL-2 50U/ml and IL-15 lng/ml. After 2 rounds of expansion, the growth and functionality of the bi-specific T cells was evaluated in vitro and in vivo.

Intracellular cytokine staining:_Bi-specific T cells (10⁵) were activated overnight with 105 LCL-OKT3, LCL, or KG1a cells in 96-well tissue culture plates, and with 10′ U251T and engineered pp65-expressing U251T cells (pp65U251T) in 24-well tissue culture plates in the presence of Brefeldin A (BD Biosciences). The cell mixture was then stained using anti-CD8, cetuximab and streptavidin, and pp65Tetramer to analyze surface co-expression of CD8, CAR and CMV-specific TCR, respectively. Cells were then fixed and permeabilized using the BD Cytofix/Cytoperm kit (BD Biosciences). After fixation, the T cells were stained with an anti-IFNγ.

CFSE Proliferation assays: Bi-specific T cells were labeled with 0.5 μM CFSE and co-cultured with stimulator cells LCL-OKT3, LCLs, and pp65 U251T for 8 days. Co-cultures with U251T and KG1a cells were used as negative controls. Proliferation of CD3- and CAR-positive populations was determined using multicolor flow cytometry.

Cytokine production assays: T cells (10⁵) were co-cultured overnight in 96-well tissue culture plates with 105 LCL-OKT3, LCL, or KG1a cells and in 24-well tissue culture plates with 105 U251T and engineered pp65-expressing U251T cells. Supernatants were then analyzed by cytometric bead array using the Bio-Plex Human Cytokine 17-Plex Panel (Bio-Rad Laboratories) according to the manufacturer's instructions.

Cytotoxicity assays: 4-hour chromium-release assays (CRA) were performed as previously described (20) using effector cells that had been harvested directly after 2 rounds of CD19 Ag stimulations.

Xenograft models: All mouse experiments were approved by the City of Hope Institutional Animal Care and Use Committee. Six- to ten-week old NOD/Scid IL-2RγC^{null} (NSG) mice were injected intravenously (i.v.) on day 0 with 2.5×10⁶ fflucGFPLCLs cells. Three days after tumor inoculation, recipient mice were injected i.v. with 2×10⁶ bi-specific T cells that had undergone 2 rounds of CD19 stimulation. To generate antigen-presenting T cells (T-APC) for vaccine, REM-expanded T cells from the autologous donor were pulsed (2 h at 37° C. in CM) with 10 μg/mL of either HLA-A2 restricted pp65 peptide (NLVPMVATV; SEQ ID NO:16), 1 ug/mL pp65 pepmix depending on whether bi-specific T cell products are pp65 tetramer dominant (GIGFVFTL (SEQ ID NO:17), donor 2) or not (pp65 pepmix donor 1) or 10 μg/mL HLA-A2 restricted control peptide specific for MP1 (GIGFVFTL; SEQ ID NO:17). Following one wash with phosphate buffered saline (PBS), 5×106 T-APC that had been irradiated with 3700 cGy were injected i.v into the T-cell-treated mice. Tumor burden was monitored with Xenogen® imaging twice a week. Human T cell engraftment in peripheral blood, bone marrow and spleen was determined by flow cytometry.

### REFERENCES

ADDIN E N. REFLIST 1. Grupp S A, Kalos M, Barrett D, Aplenc R, Porter D L, Rheingold S R, et al. Chimeric Antigen Receptor-Modified T Cells for Acute Lymphoid Leukemia. N Engl J Med. 2013; 368:1509-18.
2. Kalos M, Levine B L, Porter D L, Katz S, Grupp S A, Bagg A, et al. T cells with chimeric antigen receptors have potent antitumor effects and can establish memory in patients with advanced leukemia. Sci Transl Med. 2011; 3:95ra73.
3. Brentjens R J, Davila M L, Riviere I, Park J, Wang X, Cowell L G, et al. CD19-Targeted T Cells Rapidly Induce Molecular Remissions in Adults with Chemotherapy-Refractory Acute Lymphoblastic Leukemia. Sci Transl Med. 2013; 5:177ra38.
4. Kochenderfer J N, Dudley M E, Feldman S A, Wilson W H, Spaner D E, Maric I, et al. B-cell depletion and remissions of malignancy along with cytokine-associated toxicity in a clinical trial of anti-CD19 chimeric-antigen-receptor-transduced T cells. Blood. 2012; 119:2709-20.
5. Walter E A, Greenberg P D, Gilbert M J, Finch R J, Watanabe K S, Thomas E D, et al. Reconstitution of cellular immunity against cytomegalovirus in recipients of allogeneic bone marrow by transfer of T-cell clones from the donor. N Engl J Med. 1995; 333:1038-44.
6. Leen A M, Christin A, Myers G D, Liu H, Cruz C R, Hanley P J, et al. Cytotoxic T lymphocyte therapy with donor T cells prevents and treats adenovirus and Epstein-Barr virus infections after haploidentical and matched unrelated stem cell transplantation. Blood. 2009; 114:4283-92.
7. Bollard C M, Kuehnle I, Leen A, Rooney C M, Heslop H E. Adoptive immunotherapy for posttransplantation viral infections. Biol Blood Marrow Transplant. 2004; 10:143-55.
8. Pule M A, Savoldo B, Myers G D, Rossig C, Russell H V, Dotti G, et al. Virus-specific T cells engineered to coexpress tumor-specific receptors: persistence and antitumor activity in individuals with neuroblastoma. Nat Med. 2008; 14:1264-70.
9. Savoldo B, Rooney C M, Di Stasi A, Abken H, Hombach A, Foster A E, et al. Epstein Barr virus specific cytotoxic T lymphocytes expressing the anti-CD30zeta artificial chimeric T-cell receptor for immunotherapy of Hodgkin disease. Blood. 2007; 110:2620-30.
10. Rossig C, Bollard C M, Nuchtern J G, Rooney C M, Brenner M K. Epstein-Barr virus-specific human T lymphocytes expressing antitumor chimeric T-cell receptors: potential for improved immunotherapy. Blood. 2002; 99:2009-16.
11. Cooper L J, Al-Kadhimi Z, Serrano L M, Pfeiffer T, Olivares S, Castro A, et al. Enhanced antilymphoma efficacy of CD19-redirected influenza MP1-specific CTLs by cotransfer of T cells modified to present influenza MP1. Blood. 2005; 105:1622-31.
12. Cruz C R, Micklethwaite K P, Savoldo B, Ramos C A, Lam S, Ku S, et al. Infusion of donor-derived CD19-redirected virus-specific T cells for B-cell malignancies relapsed after allogeneic stem cell transplant: a phase 1 study. Blood. 2013; 122:2965-73.
13. van der Bij W, Speich R. Management of cytomegalovirus infection and disease after solid-organ transplantation. Clinical infectious diseases: an official publication of the Infectious Diseases Society of America. 2001; 33 Suppl 1:S32-7.
14. Soderberg-Naucler C. Does cytomegalovirus play a causative role in the development of various inflammatory diseases and cancer? Journal of internal medicine. 2006; 259:219-46.
15. La Rosa C, Longmate J, Lacey S F, Kaltcheva T, Sharan R, Marsano D, et al. Clinical evaluation of safety and immunogenicity of PADRE-cytomegalovirus (CMV) and tetanus-CMV fusion peptide vaccines with or without PF03512676 adjuvant. The Journal of infectious diseases. 2012; 205:1294-304.
16. Pelloquin F, Lamelin J P, Lenoir G M. Human B lymphocytes immortalization by Epstein-Barr virus in the presence of cyclosporin A. In Vitro Cell Dev Biol. 1986; 22:689-94.
17. Budde L E, Berger C, Lin Y, Wang J, Lin X, Frayo S E, et al. Combining a CD20 chimeric antigen receptor and an inducible caspase 9 suicide switch to improve the efficacy and safety of T cell adoptive immunotherapy for lymphoma. PLoS One. 2013; 8:e82742.
18. Cooper L J, Topp M S, Serrano L M, Gonzalez S, Chang W C, Naranjo A, et al. T-cell clones can be rendered specific for CD19: toward the selective augmentation of the graft-versus-B-lineage leukemia effect. Blood. 2003; 101:1637-44.
19. Wang X, Chang W C, Wong C W, Colcher D, Sherman M, Ostberg J R, et al. A transgene-encoded cell surface polypeptide for selection, in vivo tracking, and ablation of engineered cells. Blood. 2011; 118:1255-63.
20. Stastny M J, Brown C E, Ruel C, Jensen M C. Medulloblastomas expressing IL13Ralpha2 are targets for IL13-zetakine+ cytolytic T cells. J Pediatr Hematol Oncol. 2007; 29:669-77.
21. Jonnalagadda M, Mardiros A, Urak R, Wang X, Hoffman L J, Bernanke A, et al. Chimeric Antigen Receptors with Mutated IgG4 Fc Spacer Avoid Fc Receptor Binding and Improve T cell Persistence and Anti-Tumor Efficacy. Mol Ther. 2014; 10.1038/mt.2014.208.
22. Hudecek M, Sommermeyer D, Kosasih P L, Silva-Benedict A, Liu L, Rader C, et al. The non-signaling extracellular spacer domain of chimeric antigen receptors is decisive for in vivo antitumor activity. Cancer immunology research. 2014; 10.1158/2326-6066.cir-14-0127. Epub
23. Wang X, Berger C, Wong C W, Forman S J, Riddell S R, Jensen M C. Engraftment of human central memory-derived effector CD8+ T cells in immunodeficient mice. Blood. 2011; 117:1888-98.
24. Hinrichs C S, Borman Z A, Gattinoni L, Yu Z, Burns W R, Huang J, et al. Human effector CD8+ T cells derived from naive rather than memory subsets possess superior traits for adoptive immunotherapy. Blood. 2011; 117:808-14.
25. Marchi L F, Sesti-Costa R, Ignacchiti M D, Chedraoui-Silva S, Mantovani B. In vitro activation of mouse neutrophils by recombinant human interferon-gamma: increased phagocytosis and release of reactive oxygen species and pro-inflammatory cytokines. International immunopharmacology. 2014; 18:228-35.
26. Morgan R A, Yang J C, Kitano M, Dudley M E, Laurencot C M, Rosenberg S A. Case report of a serious adverse event following the administration of T cells transduced with a chimeric antigen receptor recognizing ERBB2. Mol Ther. 2010; 18:843-51.
27. Berger C, Jensen M C, Lansdorp P M, Gough M, Elliott C, Riddell S R. Adoptive transfer of effector CD8 T cells derived from central memory cells establishes persistent T cell memory in primates. J Clin Invest. 2008; 118:294-305.
28. Sun Q, Burton R L, Dai L J, Britt W J, Lucas K G. B lymphoblastoid cell lines as efficient APC to elicit CD8+ T cell responses against a cytomegalovirus antigen. J Immunol. 2000; 165:4105-11.
29. Leen A M, Myers G D, Sili U, Huls M H, Weiss H, Leung K S, et al. Monoculture-derived T lymphocytes specific for multiple viruses expand and produce clinically relevant effects in immunocompromised individuals. Nat Med. 2006; 12:1160-6.
30. Kalamasz D, Long S A, Taniguchi R, Buckner J H, Berenson R J, Bonyhadi M. Optimization of human T-cell expansion ex vivo using magnetic beads conjugated with anti-CD3 and Anti-CD28 antibodies. J Immunother. 2004; 27:405-18.
31. Davila M L, Riviere I, Wang X, Bartido S, Park J, Curran K, et al. Efficacy and Toxicity Management of 19-28z CAR T Cell Therapy in B Cell Acute Lymphoblastic Leukemia. Sci Transl Med. 2014; 6:224ra25.
32. Klebanoff C A, Gattinoni L, Palmer D C, Muranski P, Ji Y, Hinrichs C S, et al. Determinants of successful CD8+ T cell adoptive immunotherapy for large established tumors in mice. Clin Cancer Res. 2011; 17:5343-52.
33. Gattinoni L, Lugli E, Ji Y, Pos Z, Paulos C M, Quigley M F, et al. A human memory T cell subset with stem cell-like properties. Nat Med. 2011; 17:1290-7.
34. Aleksic M, Liddy N, Molloy P E, Pumphrey N, Vuidepot A, Chang K M, et al. Different affinity windows for virus and cancer-specific T-cell receptors: implications for therapeutic strategies. Eur J Immunol. 2012; 42:3174-9.
35. Hardwick N R, Carroll M, Kaltcheva T, Qian D, Lim D, Leong L, et al. p53MVA therapy in patients with refractory gastrointestinal malignancies elevates p53-specific CD8+ T-cell responses. Clin Cancer Res. 2014; 20:4459-70.
36. Scott A M, Wolchok J D, Old U. Antibody therapy of cancer. Nat Rev Cancer. 2012; 12:278-87.

What is claimed is:

1. A method for preparing T cells specific for cytomegalovirus (CMV) and expressing a chimeric antigen receptor (CAR), the method comprising:
(a) providing PBMC from a cytomegalovirus (CMV)-seropositive human donor;
(b) exposing the PBMC to at least one CMV antigen;
(c) selecting cells expressing IFN-γ to produce a population of cells enriched for stimulated cells specific for CMV;
(d) transducing at least a portion of the enriched population of cells with a vector expressing a CAR, the method not including either CD3 stimulation of the cells or CD28 stimulation of the cells, thereby preparing T cells specific for CMV and expressing a CAR.

2. The method of claim 1, wherein the CMV antigen is pp65 protein or an antigenic portion thereof.

3. The method of claim 1, wherein the CMV antigen comprises two or more different antigenic CMV pp65 peptides.

4. The method of claim 1, wherein the enriched population of cells is at least 40% IFN-γ positive, at least 20% CD8 positive, and at least 20% CD4 positive.

5. The method of claim 1, wherein the enriched population of cells are cultured for fewer than 10 days prior to the step of transducing the enriched population of cells with a vector encoding a CAR.

6. The method of claim 1 further comprising expanding the CMV specific T cells expressing a CAR cells by exposing them an antigen that binds to the CAR.

7. The method of claim 6, wherein the expansion takes place is the presence of at least one exogenously added interleukin.

8. A method of treating a patient suffering from cancer comprising administering a composition comprising the cells of claim 1 wherein the CAR is targeted to CD19.

9. The method of claim 8, wherein the population of human T cells are autologous to the patient.

10. The method of claim 8, wherein the population of human T cells are allogenic to the patient.

11. The method of claim 8 further comprising administering to the patient a CMV antigen.

12. The method of claim 11, wherein the step of administering a CMV antigen comprising administering T cells loaded with a CMV antigen.


SEQUENCE LISTING (15 sequences)

SEQ ID NO: 1 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 12 aa, Homo sapiens
ESKYGPPCPSCP

SEQ ID NO: 3 - protein, 22 aa, Homo sapiens
HMANPRTEINESKYGPPCPPCP

SEQ ID NO: 4-6 - skipped (3 entries)

SEQ ID NO: 7 - protein, 10 aa, synthetic construct
GGGSSGGGSG

SEQ ID NO: 8 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 129 aa, Homo sapiens
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 22 aa, Homo sapiens
MALIVLGGVAGLLLFIGLGIFF

SEQ ID NO: 11 - protein, 28 aa, Homo sapiens
MFWVLVVVGGVLACYSLLVTVAFIIFWV

SEQ ID NO: 12 - protein, 41 aa, synthetic construct
RSKRSRGGHSDYMNMTPRRPGPTRKHYQPYAPPRDFAAYRS

SEQ ID NO: 13 - protein, 42 aa, synthetic construct
KRGRKKLLYIFKQPFMRPVQTTQEEDGCSCRFPEEEEGGCEL

SEQ ID NO: 14 - protein, 41 aa, synthetic construct; identical to SEQ ID NO: 12

SEQ ID NO: 15 - protein, 22 aa, synthetic construct
ESKYGPPCPPCPGGGSSGGGSG